(19) United States
(12) Patent Application Publication
(10) Pub. No.: US 2020/0221930 A1
(43) Pub. Date: Jul. 16, 2020

(54) VISUAL ORAL SCRAPING SPOON

(71) Applicant: Xing ZHOU, Guangzhou (CN)

(72) Inventor: Xing ZHOU, Guangzhou (CN)

(21) Appl. No.: 16/832,304

(22) Filed: Mar. 27, 2020

Related U.S. Application Data
(63) Continuation of application No. PCT/CN2018/100720, filed on Aug. 15, 2018.

(30) Foreign Application Priority Data
Oct. 13, 2017 (CN) 201710957726.8

(51) Int. Cl. A61B 1/00; A61B 17/24; A61B 1/24; A61B 1/05; A61B 1/06; A61C 17/02

(52) U.S. Cl. CPC A61B 1/00087; A61B 17/244; A61B 1/24; A61B 1/00105; A61B 1/05; A61B 1/00009; A61B 1/0676; A61C 17/0202; A61B 1/00016; A61B 1/00032; A61B 1/00045

(57) ABSTRACT

A visual oral scraping spoon of the present application includes an oral scraping spoon, an oral observation instrument and a connecting mechanism. The oral scraping spoon is detachably connected to the oral observation instrument through the connecting mechanism, and under the action of an observation system of the oral observation instrument, the oral scraping spoon can be used to scrape and clean the teeth or the tongue under direct vision, and the cleaning effect can be observed in real time, and the cleaning process can be shot and photographed. Furthermore, the oral observation instrument may also be provided with a flusher, the flusher is used in conjunction with the oral scraping spoon to thoroughly flush the removed dental calculus, dental plaque, coated tongue and the like in time, and the using process is more comfortable, convenient and efficient.

### CROSS-REFERENCE TO RELATED APPLICATIONS

This application is a continuation application of PCT/CN2018/100720, entitled “VIEWING ORAL CAVITY SCRAPER” filed on Aug. 15, 2018, which claims priority to Chinese Patent Application No. 201710957726.8, entitled “VIEWING ORAL CAVITY SCRAPER” filed with the China National Intellectual Property Administration on Oct. 13, 2017, both of which are incorporated by reference in their entirety.

### FIELD OF THE INVENTION

The invention relates to a tool for oral cleaning, and particularly relates to a visual oral scraping spoon for cleaning teeth, interdental spaces and oral cavities.

### BACKGROUND OF THE INVENTION

When the teeth are not cleaned thoroughly for a long time or when people smoke, drink tea or drink alcohol for a long time, it is very easy to cause dental problems such as dental calculus, dental plaque and pigmentation on the surface of the teeth, and it is easy to cause thickening of the coated tongue. At present, the dental calculus, dental plaque, pigmentation, coated tongue and the like are usually cleaned in a professional stomatological hospital.

At present, in a tooth washing process in a hospital or a dental clinic, the common way to clean the dental calculus, dental plaque and pigmentation is to extend a work head of a tooth cleaner in high-speed rotation or vibration into the oral cavity so as to loosen the dental plaque and break the dirt on the surface of the teeth under the friction or vibration action of the work head, thereby achieving the purpose of cleaning the dental calculus, dental plaque and pigmentation.

Because the work head needs to be extended into the oral cavity and the light in the oral cavity is very dark, it is very difficult to see the condition in the oral cavity. Therefore, in the prior art, generally, the dental calculus, dental plaque, pigmentation, coated tongue and the like in the oral cavity of a patient can only be cleaned by a dentist by virtue of professional instruments and a dental treatment table or a dental treatment chair in a dental clinic.

At home, it is difficult for a patient to use the oral cleaning instruments in the prior art to clean the dental calculus, dental plaque, pigmentation and the like in the oral cavity. At present, when a coated tongue cleaner, various toothbrushes or a flusher is used to clean the teeth, dorsum and the like, because the light in the oral cavity is dark or a part to be cleaned is relatively hidden and difficult to observe, the patient can only perform these cleaning operations by feeling. Generally, the part to be cleaned cannot be cleaned, but the parts that do not need to be cleaned are cleaned repeatedly, causing excessive damage to the teeth or soft tissues in the oral cavity. Particularly, when the patient uses a tool in high-speed rotation or vibration, such as a high-speed grinding head, an ultrasonic grinding head or a dental bur, to clean the oral cavity, once the work head deviates from the work part, it is very easy to cause accidental injury.

In order to overcome the defects of the prior art, it is necessary to develop a tool which can be used by the patient at home to carry out targeted cleaning the teeth or coated tongue by using a display screen of a mobile phone in a visual state.

### SUMMARY

The visual oral scraping spoon of the present application is capable of cleaning the teeth and coated tongue in a visual state by utilizing a special cleaning tool, thereby effectively solving the oral problems of dental calculus, dental plaque, pigmentation, excessively thick coated tongue and the like. Furthermore, the cleaning process and the cleaning effect are directly observed by the oral observation instrument 200.

The visual oral scraping spoon of the present application is characterized in that the visual oral scraping spoon 900 includes an oral scraping spoon 100, an oral observation instrument 200 and a connecting mechanism 300;

A. the oral scraping spoon 100 includes a scraping plate 11, an assembling and disassembling mechanism 12 and a bracket 13; the scraping plate 11 is disposed at a front end of the bracket 13; the assembling and disassembling mechanism 12 for mounting the oral scraping spoon 100 on the oral observation instrument 200 or disassembling the oral scraping spoon 100 from the oral observation instrument 200 is disposed on the bracket 13;

B. the oral observation instrument 200 includes a housing 21, a power system 22, an illuminating system 23, an observation system 24, a circuit system 25 and a switch 26; the illuminating system 23, the observation system 24, the circuit system 25 and the power system 22 are mounted in the housing 21, and the switch 26 is mourned on the housing 21; the illuminating system 23, the observation system 24, the power system 22 and the switch 26 are connected together through the circuit system 25;

C. the connecting mechanism 300 includes the assembling and disassembling mechanism 12; the oral scraping spoon 100 is mounted on the oral observation instrument 200 through the connecting mechanism 300.

During use, the oral scraping spoon 100 is mounted on the oral observation instrument 200 through the connecting mechanism 300, the oral scraping spoon 100 can be used to clean the dental calculus, dental plaque or pigmentation on the surface of the teeth under direct vision through the illuminating system 23 and the observation system 24 of the oral observation instrument 200, the oral scraping spoon 100 can also be used to clean the coated tongue on the dorsum, and meanwhile, the cleaning process and the cleaning effect can be observed in real time through the oral observation instrument 200.

The scraping plate 11 is within the field of view of the observation system 24 of the oral observation instrument 200, thereby ensuring that in an operation process, the working position of the scraping plate 11 can be clearly seen to effectively avoid accidental injury.

The scraping plate 11 of the oral scraping spoon 100 has a radian 11-4 meeting the requirements of a human oral cavity, thereby being more convenient for cleaning the teeth or the tongue.

The assembling and disassembling mechanism 12 is a positioning block or a positioning groove of a concave-convex clamping connecting mechanism, or a bolt or a latch of a lock button connecting mechanism, or a sleeve or a positioning rod of an interference fit connecting mechanism, or a nut or a screw of a rotation adjustment connecting mechanism. Herein, the applicant only enumerates the above several detachable connection modes, and those skilled in the art may design different connection modes according to actual requirements in combination with the prior art without departing from the scope of protection of the present application.

The assembling and disassembling mechanism 12 is a T-shaped positioning block 12-1 for connecting to the oral observation instrument 200.

The connecting mechanism 300 is a detachable mechanical connecting mechanism; the connecting mechanism 300 may be an independent component which can be disposed on the oral scraping spoon 100 or disposed on the oral observation instrument 200; or a portion of the detachable mechanical connecting mechanism is disposed on the oral scraping spoon 100, and the other portion of the detachable mechanical connecting mechanism is disposed on the oral observation instrument 200 to form a combined mechanical connecting mechanism. By virtue of a detachable connection mode, the oral scraping spoon 100 can be replaced, cleaned and stored very conveniently.

The mechanical connecting mechanism includes concave-convex clamping connection, or slide groove connection, or pin connection, or key connection, or threaded connection, or screw connection, or buckle connection, or hook connection, or interference fit connection. Herein, the applicant only enumerates the above several connection modes, and those skilled in the art may design different connection modes according to actual requirements in combination with the prior art without departing from the scope of protection of the present application.

The housing 21 of the oral observation instrument 200 is provided with a positioning groove 21-1 and a clamping block 21-2; the positioning block 12-1 of the oral scraping spoon 100 can be embedded in the positioning groove 21-1, and the clamping block 21-2 can prevent the bracket 13 of the oral scraping spoon 100 from sliding backward; the positioning block 12-1, the positioning groove 21-1 and the clamping block 21-2 cooperate to form the connecting mechanism 300.

The observation system 24 is a camera system 24-1, and the camera system 24-1 includes a camera 24-1-1, a data processing and output system 24-1-2, a display 24-1-3, a circuit system 25 and a power system 22.

Video data output by the data processing and output system 24-1-2 of the camera system 24-1 can be displayed on a display 24-1-3 in a mode of wired connection or wireless connection, and the display 24-1-3 includes a smart phone 24-1-31, or a computer 24-1-32, or a liquid crystal display 24-1-33, or a television 24-1-34.

The illuminating system 23 is disposed near the camera 24-1-1. In order to increase illumination, more LED lamps may also be disposed on the housing 21 of the oral observation instrument 200 so as to adjust the illumination brightness.

The oral observation instrument 200 is of a waterproof design. The waterproof design is more convenient for operation and cleaning.

The oral observation instrument 200 is also provided with a flusher 404 capable of flushing the oral cavity; the flusher 404 includes a sprayer 404-1, a water tank 404-2, a water pipe 404-3, a water spraying switch 404-4 and a pressurizing device 404-5; the water tank 404-2 and the pressurizing device 404-5 are mounted on the housing 21 of the oral observation instrument 200; the sprayer 11 is mounted near the observation system 24 at the front end 200-1 of the oral observation instrument 200. When the flusher 404 is mounted on the oral observation instrument 200, in a process of cleaning the teeth or the tongue by using the oral scraping spoon 100, the flusher 404 is used to spray water to flush a work part to wash away the scraped dental calculus, dental plaque, coated tongue and the like in time, so that the cleaning process is more comfortable and convenient.

The pressurizing device 404-5 is an electric pressurizing device 404-51.

The electric pressurizing device 404-51 is an electric air pressurizing device 404-51-1 or an electric water pressurizing device 404-51-2.

The electric water pressurizing device 404-51-2 is a submersible pump 404-51-21 or a water pump 404-51-22.

The sprayer 404-1 of the flusher 404 can be built in the housing 21 of the front end 200-1 of the oral observation instrument 200.

The flusher 404 can be used in conjunction with the oral scraping spoon 100.

A direction of a water column sprayed by the flusher 404 is substantially the same as a direction of the scraping plate 11 of the oral scraping spoon 100, so that the dirt can be scraped and simultaneously washed away in time, and the cleaning effect can be conveniently observed.

The visual oral scraping spoon 900 of the present application includes the oral scraping spoon 100, the oral observation instrument 200 and the connecting mechanism 300. The oral scraping spoon 100 is connected to the oral observation instrument 200 through the connecting mechanism 300, and under the action of the observation system 24 of the oral observation instrument 200, the oral scraping spoon 100 can be used to scrape and clean the teeth or the tongue under direct vision, so that the cleaning effect can be observed in real time, and the cleaning process can be shot and photographed. Furthermore, the oral observation instrument 200 may also be provided with the flusher 404, and the flusher 404 is used in conjunction with the oral scraping spoon 100 to thoroughly flush the cleaned dental calculus, dental plaque, coated tongue and the like in time, so that the using process is more comfortable, convenient and efficient.

### BRIEF DESCRIPTION OF THE DRAWINGS

FIG. 1 is a schematic perspective view of a visual oral scraping spoon, made of a rigid material, of the present application.

FIG. 1-1 is an exploded view of FIG. 1.

FIG. 1-2 is a cross-sectional view along A-A of FIG. 1.

FIG. 1-3 is a schematic perspective view of a scraping spoon of FIG. 1.

FIG. 2 is a schematic perspective view of a visual oral scraping spoon, made of a flexible material, of the present application.

FIG. 2-1 is an exploded view of FIG. 2.

FIG. 3 is a working principle diagram of the visual oral scraping spoon of the present application connected to a display in a wireless mode.

FIG. 4 is a working principle diagram of the visual oral scraping spoon of the present application connected to a display in a wired mode.

FIG. 5 is a schematic perspective view of the visual oral scraping spoon including a flusher of the present application.

FIG. 5-1 is an exploded view of FIG. 5.

FIG. 5-2 is a cross-sectional view along B-B of FIG. 5.

In the above drawings:

oral scraping spoon 100, oral observation instrument 200, connecting mechanism 300, visual oral scraping spoon 900 of the present application.

On Oral Scraping Spoon:

scraping plate 11, assembling and disassembling mechanism 12, bracket 13;

radian 11-4 of scraping plate;

positioning block 12-1.

On Oral Observation Instrument:

housing 21, power system 22, illuminating system 23, observation system 24, circuit system 25, switch 26;

positioning groove 21-1 for connecting oral scraping spoon, clamping block 21-2 for fixing oral scraping spoon;

camera system 24-1;

camera 24-1-1, data processing and output system 24-1-2, display 24-1-3;

smart phone 24-1-31, computer 24-1-32, liquid crystal display/television 24-1-33, tablet computer 24-1-34;

flusher 404, sprayer 404-1, water tank 404-2, water pipe 404-3, water spraying switch 404-4, pressurizing device 404-5, water filling hole 404-6;

electric pressurizing device 404-51, pressurizing device switch 404-52.

electric air pressurizing device 404-51-1, electric air compressor 404-51-11, air inlet system 404-51-111, air compressor 404-51-112, air filling pipe 404-51-113.

### DESCRIPTION OF EMBODIMENTS

### Embodiment 1: Visual Oral Scraping Spoon of the Present Application

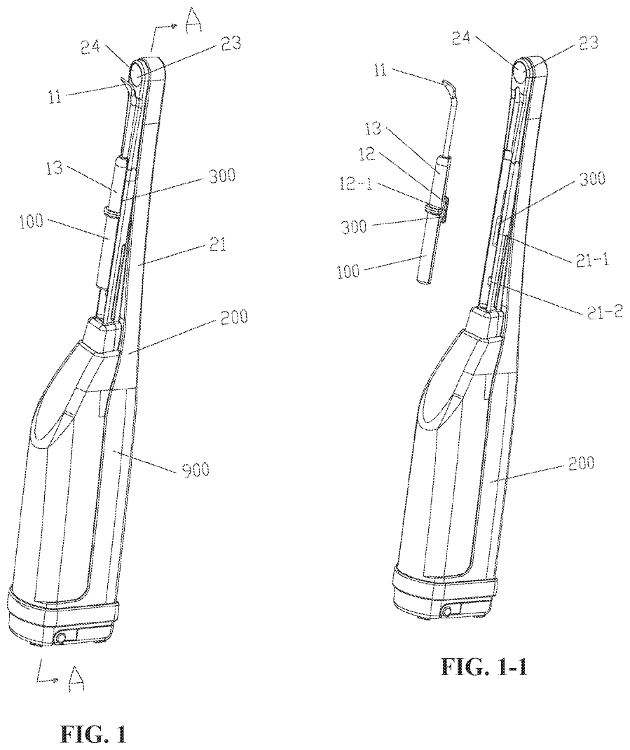

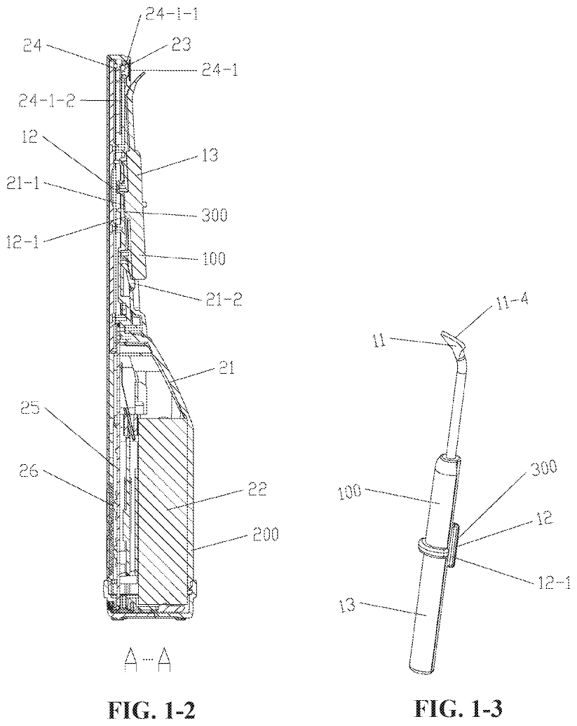

Referring to FIG. 1 to FIG. 1-3, a visual oral scraping spoon 900 of the present embodiment includes an oral scraping spoon 100, an oral observation instrument 200 and a connecting mechanism 300.

Referring to FIG. 1 and FIG. 1-1, the oral scraping spoon 100 includes a scraping plate 11, an assembling and disassembling mechanism 12 and a bracket 13. The scraping plate 11 is disposed at a front end of the bracket 13. The assembling and disassembling mechanism 12 for mounting the oral scraping spoon 100 on the oral observation instrument 200 or disassembling the oral scraping spoon 100 from the oral observation instrument 200 is disposed on the bracket 13.

The oral observation instrument 200 includes a housing 21, a power system 22, an illuminating system 23, an observation system 24, a circuit system 25 and a switch 26. The illuminating system 23, the observation system 24, the circuit system 25 and the power system 22 are mounted in the housing 21, and the switch 26 is mounted on the housing 21. The illuminating system 23, the observation system 24, the power system 22 and the switch 26 are connected together through the circuit system 25.

The connecting mechanism 300 includes the assembling and disassembling mechanism 12, and the oral scraping spoon 100 is mounted on the oral observation instrument 200 through the connecting mechanism 300.

The scraping plate 11 is within the field of view of the observation system 24 of the oral observation instrument 200, thereby ensuring that in an operation process, the working position of the scraping plate 11 can be clearly seen to effectively avoid accidental injury.

Referring to FIG. 1-3 and FIG. 2-1, the scraping plate 11 of the oral scraping spoon 100 has a radian 11-4 meeting the requirements of a human oral cavity, thereby being more convenient for cleaning the teeth or the tongue.

Referring to FIG. 1-1 and FIG. 1-3, in the present embodiment, the assembling and disassembling mechanism 12 is a positioning block or a positioning groove of a concave-con vex clamping connecting mechanism. The assembling and disassembling mechanism 12 is a T-shaped positioning block 12-1 for connecting to the oral observation instrument 200.

The assembling and disassembling mechanism 12 may also be a boll or a latch of a lock button connecting mechanism, or a sleeve or a positioning rod of an interference fit connecting mechanism, or a nut or a screw of a rotation adjustment connecting mechanism. Herein, the applicant only enumerates the above several detachable connection modes, and those skilled in the art may design different connection modes according to actual requirements in combination with the prior art without departing from the scope of protection of the present application.

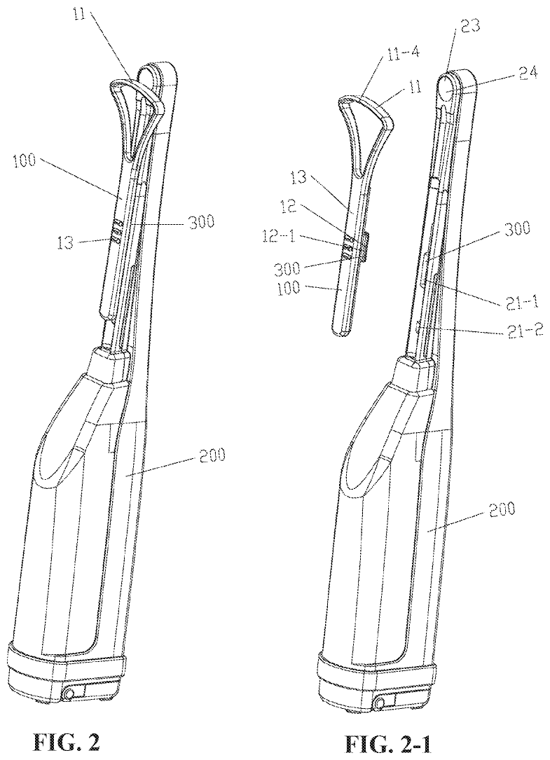

Referring to FIG. 1-1 and FIG. 2-1, in the present embodiment, the connection mechanism 300 is a detachable mechanical connecting mechanism. A portion of the detachable mechanical connecting mechanism is disposed on the oral scraping spoon 100, and the other portion of the detachable mechanical connecting mechanism is disposed on the oral observation instrument 200 to form a combined mechanical connecting mechanism. By virtue of a detachable connection mode, the oral scraping spoon 100 can be replaced, cleaned and stored very conveniently.

Furthermore, the connecting mechanism 300 may also be an independent component which can be disposed on the oral scraping spoon 100 or disposed on the oral observation instrument 200.

In the present embodiment, the mechanical connecting mechanism is in concave-convex clamping connection. The housing 21 of the oral observation instrument 200 is provided with a positioning groove 21-1 and a clamping block 21-2; the positioning block 12-1 of the oral scraping spoon 100 can be embedded in the positioning groove 21-1, and the clamping block 21-2 can prevent the bracket 13 of the oral scraping spoon 100 from sliding backward; the positioning block 12-1, the positioning groove 21-1 and the clamping block 21-2 cooperate to form the connecting mechanism 300.

Furthermore, the mechanical connecting mechanism may also be slide groove connection, or pin connection, or key connection, or threaded connection, or screw connection, or buckle connection, or hook connection, or interference fit connection. Herein, the applicant only enumerates the above several connection modes, and those skilled in the art may design different connection modes according to actual requirements in combination with the prior art without departing from the scope of protection of the present application.

In the present embodiment, the observation system 24 is a camera system 24-1, and the camera system 24-1 includes a camera 24-1-1, a data processing and output system 24-1-2, a display 24-1-3, a circuit system 25 and a power system 22.

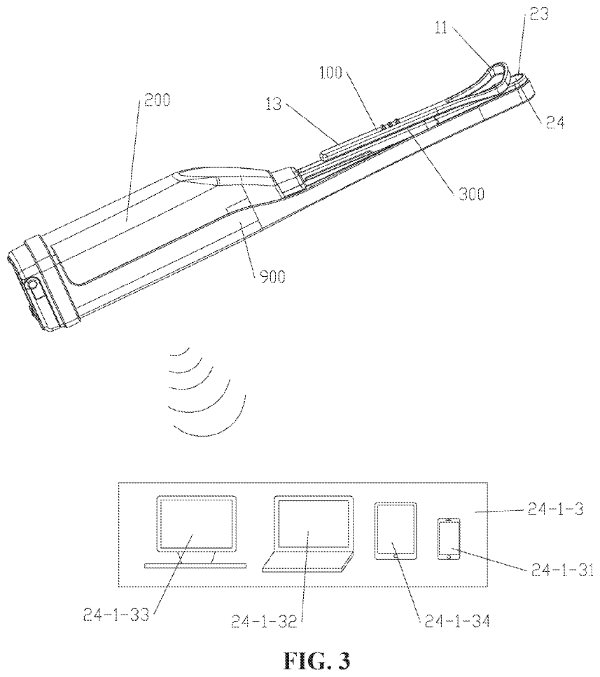

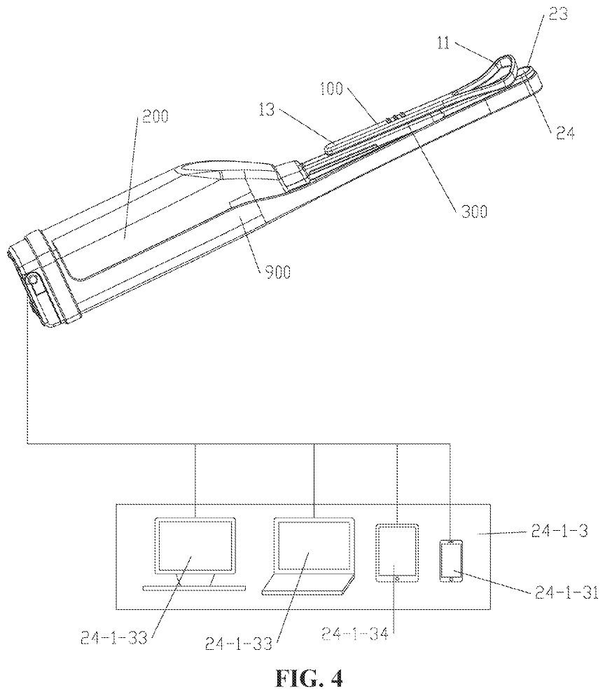

Referring to FIG. 3 and FIG. 4, video data output by the data processing and output system 24-1-2 of the camera system 24-1 can be displayed on the display 24-1-3 in a mode of wired connection or wireless connection, and the display 24-1-3 includes a smart phone 24-1-31, or a computer 24-1-32, or a liquid crystal display 24-1-33, or a television 24-1-34.

In the present embodiment, the illuminating system 23 is disposed near the camera 24-1-1 and can directly supply illumination for the camera 24-1-1 so as to improve the shooting effect of the camera 24-1-1. In order to increase illumination, more LED lamps may also be disposed on other parts on the housing 21 of the oral observation instrument 200 so as to adjust the illumination brightness.

The oral observation instrument 200 is of a waterproof design which is more convenient for operation and cleaning.

When the visual oral scraping spoon of the present application is used to clean flexible tissues, such as coated tongue and gingivae, in the oral cavity, the scraping plate 11 is made of a flexible material, thereby better ensuring the comfort during the cleaning process.

When the visual oral scraping spoon of the present application is used for cleaning the surface of the teeth, such as the dental calculus and dental plaque, the scraping plate 11 may be made of a rigid material, such as stainless steel, titanium alloy and polycarbonate (PC), and the scraping plate 11 can be designed into different shapes as needed and can be made of a corresponding material accordingly.

During use, the oral scraping spoon 100 is mounted on the oral observation instrument 200 through the connecting mechanism 300, the oral scraping spoon 100 can be used to clean the dental calculus, dental plaque or pigmentation on the surface of the teeth under direct vision through the illuminating system 23 and the observation system 24 of the oral observation instrument 200, the oral scraping spoon 100 can also be used to clean the coated tongue on the dorsum, meanwhile, the cleaning process and the cleaning effect can be observed in real time through the oral observation instrument 200, and the cleaning process is shot and photographed.

### Embodiment 2: Visual Oral Scraping Spoon Including Flusher of the Present Application

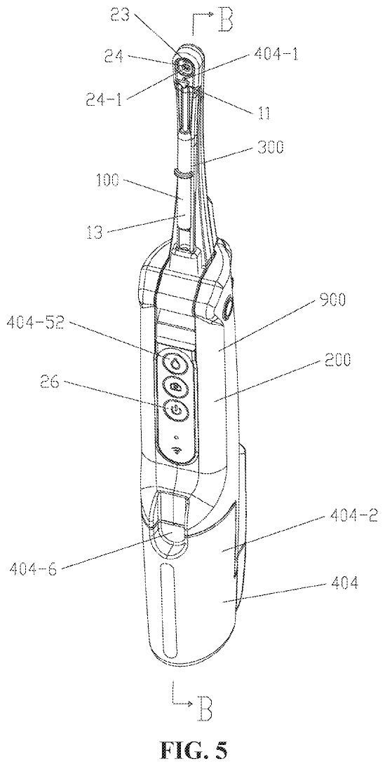

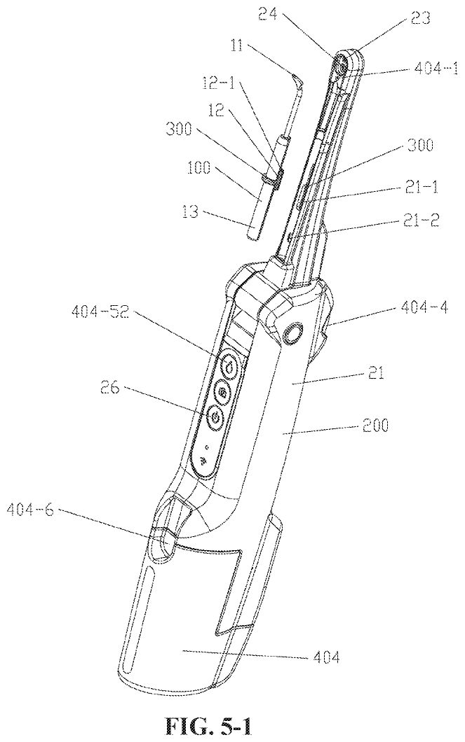

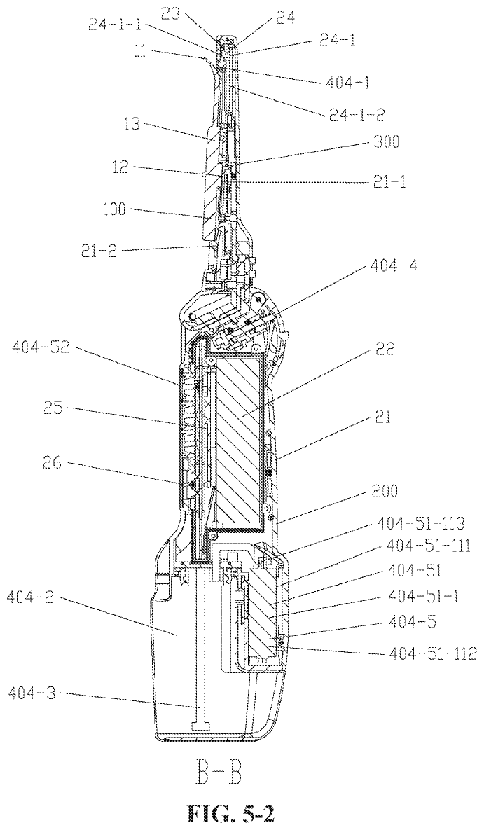

Referring to FIG. 5 to FIG. 5-2, the difference between the present embodiment and Embodiment 1 lies in that: in the present embodiment, the oral observation instrument 200 is also provided with a flusher 404 capable of flushing the oral cavity.

Referring to FIG. 5-2, the flusher 404 includes a sprayer 404-1, a water tank 404-2, a water pipe 404-3, a water spraying switch 404-4 and a pressurizing device 404-5. The water tank 404-2 and the pressurizing device 404-5 are mounted in the housing 21 of the oral observation instrument 200. The sprayer 404-1 is mounted near the observation system 24 at the front end 200-1 of the oral observation instrument 200.

In the present embodiment, the pressurizing device 404-5 is an electric pressurizing device 404-51. Due to the electric pressurizing design, in a using process, the water in the water tank 404-2 can be automatically pressurized by just turning on the switch, so that the using process is more convenient. Of course, a designer may also choose a manual pressurizing device without departing from the scope of protection of the present application.

In the present embodiment, the electric pressurizing device 404-51 is an electric air pressurizing device 404-51-1. The electric pressurizing device 404-51 may also be an electric water pressurizing device.

In the present embodiment, the electric air pressurizing device 404-51-1 is an air compressor 404-51-11.

Referring to FIG. 5-2, the electric air compressor 404-51-11 includes an air inlet system 404-51-111, a compressor 404-51-112 and an air filling pipe 404-51-113.

In the present embodiment, the sprayer 404-1 of the flusher 404 can be built in the housing 21 of the front end 200-1 of the oral observation instrument 200, thus when the flusher 404 and the oral scraping spoon 100 need to be used together, the oral scraping spoon 100 can be conveniently mounted on the oral observation instrument 200, and water is directly sprayed by the built-in sprayer 404-1 so as to clean the oral cav n conjunction with the oral scraping spoon 100.

During use, the oral scraping spoon 100 is mounted on the oral observation instrument 200 through the connecting mechanism 300, the oral scraping spoon 100 can be used to clean the dental calculus, dental plaque or pigmentation on the surface of the teeth under direct vision through the illuminating system 23 and the observation system 24 of the oral observation instrument 200, or the oral scraping spoon 100 is used to clean the coated tongue on the dorsum. In the cleaning process, when it is necessary to spray water to flush the part to be cleaned, the pressurizing device switch 404-52 is turned on, the electric air compressor 404-51-11 pressurizes water in the water tank 404-2, after pressurization, the water spraying switch 404-4 is turned on, and the pressurized water in the water tank 404-2 is sprayed by the sprayer 404-1 through the water pipe 404-3 so as to flush the part to be cleaned to wash away the scraped dental calculus, dental plaque, coated tongue and the like in time, so that the cleaning process is more comfortable and convenient. When it is necessary to replenish water into the water tank 404-2, the water filling hole 404-6 formed in the water tank 404-2 can be opened to replenish water into the water tank.

It should be noted that the structures disclosed and described herein may be replaced with other structures having the same effect, and the embodiments described herein are not the only structures that can implement the invention. Although the preferred embodiments of the present application have been described herein, it is apparent to those skilled in the art that the embodiments are merely examples, and various variations, modifications, and replacements may be made by those skilled in the art without departing from the invention. Therefore, the protection scope of the present application should be subject to the spirit and scope of the appended claims of the present application.

1. A visual oral scraping spoon, comprising an oral scraping spoon, an oral observation instrument and a connecting mechanism, wherein:
A. the oral scraping spoon comprises a scraping plate, an assembling and disassembling mechanism and a bracket; the scraping plate is disposed at a front end of the bracket; the assembling and disassembling mechanism for mounting the oral scraping spoon on the oral observation instrument or disassembling the oral scraping spoon from the oral observation instrument is disposed on the bracket;
B. the oral observation instrument comprises a housing, a power system, an illuminating system, an observation system, a circuit system and a switch; the illuminating system, the observation system, the circuit system and the power system are mounted in the housing, and the switch is mounted on the housing; the illuminating system, the observation system, the power system and the switch are connected together through the circuit system; and
C. the connecting mechanism comprises the assembling and disassembling mechanism; the oral scraping spoon is mounted on the oral observation instrument through the connecting mechanism.

2. The visual oral scraping spoon according to claim 1, wherein the scraping plate is within the field of view of the observation system of the oral observation instrument.

3. The visual oral scraping spoon according to claim 1, wherein the scraping plate of the oral scraping spoon has a radian meeting the requirements of a human oral cavity.

4. The visual oral scraping spoon according to claim 1, wherein the assembling and disassembling mechanism is a positioning block or a positioning groove of a concave-convex clamping connecting mechanism, or a bolt or a latch of a lock button connecting mechanism, or a sleeve or a positioning rod of an interference fit connecting mechanism, or a nut or a screw of a rotation adjustment connecting mechanism.

5. The visual oral scraping spoon according to claim 4, wherein the assembling and disassembling mechanism is a T-shaped positioning block for connecting to the oral observation instrument.

6. The visual oral scraping spoon according to claim 1, wherein the connecting mechanism is a detachable mechanical connecting mechanism; the connecting mechanism may be an independent component which may be disposed on the oral scraping spoon or disposed on the oral observation instrument; or a portion of the detachable mechanical connecting mechanism is disposed on the oral scraping spoon, and the other portion of the detachable mechanical connecting mechanism is disposed on the oral observation instrument to form a combined mechanical connecting mechanism.

7. The visual oral scraping spoon according to claim 6, wherein the mechanical connecting mechanism comprises concave-convex clamping connection, or slide groove connection, or pin connection, or key connection, or threaded connection, or screw connection, or buckle connection, or hook connection, or interference fit connection.

8. The visual oral scraping spoon according to claim 1, wherein the housing of the oral observation instrument is provided with a positioning groove and a clamping block; the positioning block of the oral scraping spoon can be embedded in the positioning groove, and the clamping block can prevent the bracket of the oral scraping spoon from sliding backward; the positioning block, the positioning groove and the clamping block cooperate to form the connecting mechanism.

9. The visual oral scraping spoon according to claim 1, wherein the observation system is a camera system, and the camera system comprises a camera, a data processing and output system, a display, a circuit system and a power system.

10. The visual oral scraping spoon according to claim 9, wherein video data output by the data processing and output system of the camera system can be displayed on a display in a mode of wired connection or wireless connection, and the display comprises a smart phone, or a computer, or a liquid crystal display, or a television.

11. The visual oral scraping spoon according to claim 9, wherein the illuminating system is disposed near the camera.

12. The visual oral scraping spoon according to claim 1, wherein the oral observation instrument is of a waterproof design.

13. The visual oral scraping spoon according to claim 1, wherein the oral observation instrument is also provided with a flusher capable of flushing the oral cavity; the flusher comprises a sprayer, a water tank, a water pipe, a water spraying switch and a pressurizing device; the water tank and the pressurizing device are mounted on the housing of the oral observation instrument; the sprayer is mounted near the observation system at the front end of the oral observation instrument.

14. The visual oral scraping spoon according to claim 13, wherein the pressurizing device is an electric pressurizing device.

15. The visual oral scraping spoon according to claim 14, wherein the electric pressurizing device is an electric air pressurizing device or an electric water pressurizing device.

16. The visual oral scraping spoon according to claim 14, wherein the electric water pressurizing device is a submersible pump or a water pump.

17. The visual oral scraping spoon according to claim 13, wherein the sprayer of the flusher can be built in the housing of the front end of the oral observation instrument.

18. The visual oral scraping spoon according to claim 13, wherein the flusher can be used in conjunction with the oral scraping spoon.

19. The visual oral scraping spoon according to claim 18, wherein a direction of a water column sprayed by the flusher is substantially the same as a direction of the scraping plate of the oral scraping spoon.
